(19) United States
(12) Patent Application Publication
(10) Pub. No.: US 2006/0128803 A1
(43) Pub. Date: Jun. 15, 2006

(54) Method of treating dry eye disorders using 13(S)-HODE and its analogs

(75) Inventors: Peter G. Klimko, Fort Worth, TX (US); Daniel A. Gamache, Arlington, TX (US); Gustav Graff, Cleburne, TX (US); Mark R. Hellberg, Arlington, TX (US); John R. Falck, Dallas, TX (US); John M. Yanni, Burleston, TX (US)

Correspondence Address: Alcon Research, Ltd.;Patrick M. Ryan(Q-148), IP Legal Department, 6201 So. Freeway, Fort Worth, TX, 76134-2099, US

(73) Assignee: Alcon, Inc.

(21) Appl. No.: 11/302,836

(22) Filed: Dec. 14, 2005

Related U.S. Application Data
(60) Provisional application No. 60/635,743, filed on Dec. 14, 2004.

(51) Int. Cl. A61K 31/202

(52) U.S. Cl. 514/559; 514/560

(57) ABSTRACT

The topical use of 13(S)-HODE and analogs are disclosed for the treatment of dry eye disorders.

This application claims priority to U.S. Provisional application, U.S. Ser. No. 60/635,743 filed Dec. 14, 2004.

The present invention is directed to the treatment of dry eye disorders. In particular, the present invention is directed toward the use of 13(S)-HODE and its analogs to treat dry eye in mammals.

### BACKGROUND OF THE INVENTION

Dry eye, also known generically as keratoconjunctivitis sicca, is a common ophthalmological disorder affecting millions of Americans each year. The condition is particularly widespread among post-menopausal women due to hormonal changes following the cessation of fertility. Dry eye may afflict an individual with varying severity. In mild cases, a patient may experience burning, a feeling of dryness, and persistent irritation such as is often caused by small bodies lodging between the eye lid and the eye surface. In severe cases, vision may be substantially impaired. Other diseases, such as Sjogren's disease and cicatricial pemphigoid manifest dry eye complications.

Although it appears that dry eye may result from a number of unrelated pathogenic causes, all presentations of the complication share a common effect, that is the breakdown of the pre-ocular tear film, which results in dehydration of the exposed outer surface and many of the symptoms outlined above (Lemp, Report of the National Eye Institute/lndustry Workshop on Clinical Trials in Dry Eyes, The CLAO Journal, volume 21, number 4, pages 221-231 (1995)).

Practitioners have taken several approaches to the treatment of dry eye. One common approach has been to supplement and stabilize the ocular tear film using so-called artificial tears instilled throughout the day. Other approaches include the use of ocular inserts that provide a tear substitute or stimulation of endogenous tear production.

Examples of the tear substitution approach include the use of buffered, isotonic saline solutions, aqueous solutions containing water soluble polymers that render the solutions more viscous and thus less easily shed by the eye. Tear reconstitution is also attempted by providing one or more components of the tear film such as phospholipids and oils. Phospholipid compositions have been shown to be useful in treating dry eye; see, e.g., McCulley and Shine, Tear film structure and dry eye, Contactologia, volume 20(4), pages 145-49 (1998); and Shine and McCulley, Keratoconjunctivitis sicca associated with meibomian secretion polar lipid abnormality, Archives of Ophthalmology, volume 116(7), pages 849-52 (1998). Examples of phospholipid compositions for the treatment of dry eye are disclosed in U.S. Pat. Nos. 4,131,651 (Shah et al.), 4,370,325 (Packman), 4,409,205 (Shively), 4,744,980 and 4,883,658 (Holly), 4,914,088 (Glonek), 5,075,104 (Gressel et al.), 5,278,151 (Korb et al.), 5,294,607 (Glonek et al.), 5,371,108 (Korb et al.) and 5,578,586 (Glonek et al.). U.S. Pat. No. 5,174,988 (Mautone et al.) discloses phospholipid drug delivery systems involving phospholipids, propellants and an active substance.

Another approach involves the provision of lubricating substances in lieu of artificial tears. For example, U.S. Pat. No. 4,818,537 (Guo) discloses the use of a lubricating, liposome-based composition, and U.S. Pat. No. 5,800,807 (Hu et al.) discloses compositions containing glycerin and propylene glycol for treating dry eye.

Although these approaches have met with some success, problems in the treatment of dry eye nevertheless remain. The use of tear substitutes, while temporarily effective, generally requires repeated application over the course of a patient's waking hours. It is not uncommon for a patient to have to apply artificial tear solution ten to twenty times over the course of the day. Such an undertaking is not only cumbersome and time consuming, but is also potentially very expensive. Transient symptoms of dry eye associated with refractive surgery have been reported to last in some cases from six weeks to six months or more following surgery.

Aside from efforts directed primarily to the alleviation of symptoms associated with dry eye, methods and compositions directed to treatment of the dry eye condition have also been pursued. For example, U.S. Pat. No. 5,041,434 (Lubkin) discloses the use of sex steroids, such as conjugated estrogens, to treat dry eye conditions in post-menopausal women; U.S. Pat. No. 5,290,572 (MacKeen) discloses the use of finely divided calcium ion compositions to stimulate pre-ocular tear film production; and U.S. Pat. No. 4,966,773 (Gressel et al.) discloses the use of microfine particles of one or more retinoids for ocular tissue normalization.

Some recent literature reports suggest that patients suffering from dry eye syndrome disproportionately exhibit the hallmarks of excessive inflammation in relevant ocular tissues, such as the lacrimal and meibomian glands. The use of various compounds to treat dry eye patients, such as steroids [e.g. U.S. Pat. No. 5,958,912; Marsh, et al., Topical nonpreserved methyiprednisolone therapy for keratoconjunctivitis sicca in Siogren syndrome, Ophthalmology, 106(4): 811-816 (1999); Pflugfelder, et. al. U.S. Pat. No. 6,153,607], cytokine release inhibitors (Yanni, J. M.; et. al. WO 0003705 A1), cyclosporine A [Tauber, J. Adv. Exp. Med. Biol. 1998, 438 (Lacrimal Gland, Tear Film, and Dry Eye Syndromes 2), 969], and 15-HETE (Yanni et. al., U.S. Pat. No. 5,696,166), has been disclosed.

13S-HODE [(13S)-(9Z,11E)-octadeca-9,11-enoic acid] is the major endogenous product of the 15-lipoxygenase-1 enzyme catalyzed lipoxygenation of linoleic acid. It has been reported to inhibit cancer cell proliferation [see for example: Prostaglandins, Leukotrienes and Essential Fatty Acids 2004, 70(1), 7-15; Carcinogenesis 2003 Feb; 24(2), 243-7] and tumor cell adhesion to endothelial cells [Cancer Res. 1989, 49(4), 1029-37]. 13S-HODE has also been shown to inhibit the hyperproliferation of mouse skin epidermal cells in a psoriasis model [Prostaglandins Leukot Essent Fatty Acids. 2000, 62(1), 13-9]. These anti-proliferative effects have been associated with a suppression of protein kinase C activity [Proc Natl Acad Sci U S A. 1995, 92(20), 9323-7; J Invest Dermatol. 1999, 112(1), 42-8;] and a lowering of nuclear MAP kinase concentration [Cell Signal. 1998, 10(2), 143-9]. It is also reportedly a pM agonist of the PPAR-γ receptor, possibly serving to induce/maintain colorectal cell differentiation [Carcinogenesis 2003, 24(11), 1717-1722]. Additionally, 13S-HODE reportedly is produced by endothelial cells to inhibit platelet adhesion to blood vessel walls [J Biol Chem. 1985 260(30), 16056-9] and to inhibit vascular cell wall hyperplasia following injury (WO 2001076568 A2). 13S-HODE and certain analogs (e.g., 13-KODE, the 13-ketone analog of 13-HODE) have been claimed as aromatase inhibitors to treat estrogen-dependent breast cancer (U.S. Pat. No. 5,102,912).

### SUMMARY OF THE INVENTION

The present invention is directed to methods for the treatment of dry eye. According to the methods of the present invention, 13(S)-HODE or an analog of 13(S)-HODE is administered to a patient. The 13(S)-HODE or analog is preferably administered in an ophthalmic composition dosed topically to a patient's eye.

### DETAILED DESCRIPTION OF THE INVENTION

Unless indicated otherwise, all component amounts are presented on a % (w/v) basis.

According to the methods of the present invention, a composition comprising a compound of formula I is topically administered to a mammal in need thereof: 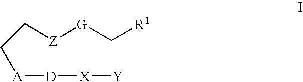
wherein

R¹ is CO₂R, CH₂OR², CONR³R⁴, or CO₂⁻R⁺;

R is H, C₁-C₆ alkyl, C₃-C₆ alkenyl, C₃-C₆ alkynyl, C₃-C₆ cycloalkyl, or phenyl;

R⁺ is Li⁺, Na⁺, K⁺, or an ammonium moiety of formula ⁺NR⁵R⁶R⁷R⁸, where R⁵, R⁶, R⁷, and R⁸ are independently H or C₁-C₆ alkyl, each alkyl group optionally bearing an OH or OCH₃ substituent;

R² is H, C(O)R⁹, C₁-C₆ alkyl, C₃-C₆ alkenyl, C₃-C₆ alkynyl, C₃-C₆ cycloalkyl, benzyl, or phenyl, where R⁹ is H, C₁₋₆ alkyl, C₃-C₆ alkenyl, C₃-C₆ alkynyl, C₃-C₆ cycloalkyl, benzyl, or phenyl;

R³ and R⁴ are independently H, C₁-C₆ alkyl, C₃-C₆ alkenyl, C₃-C₆ alkynyl, C₃-C₆ cycloalkyl, H, benzyl, phenyl, OH, OCH₃, or OC₂H₅, provided that at most only one of R³ and R⁴ is OH, OCH₃, or OC₂H₅;

G is CH₂, O, or S;

Z is CH₂CH═CH, CH═CHCH₂, CH₂C≡C, C≡CCH₂, (CH₂)₃, or CH═C═CH when G is CH₂, and is CH═CHCH₂, C≡CCH₂, or (CH₂)₃ when G is O or S;

A and D are independently CH₂CH₂, CH═CH, or C≡C, provided that if A is CH₂CH₂, then D is not CH₂CH₂;

X is C═O or CR¹⁰R₁₁;

R¹⁰ is H or CH₃;

R¹¹ is H, C₁-C₆ alkyl, C₃-C₆ alkenyl, C₃-C₆ alkynyl, C₃-C₆ cycloalkyl, phenyl, benzyl, or C(O)R¹², where R¹² is H, C₁-C₆ alkyl, C₃-C₆ alkenyl, C₃-C₆ alkynyl, C₃-C₆ cycloalkyl, phenyl, or benzyl;

Y is n-C₅H₁₁, C(CH₃)H-n-C₄H₉, C(CH₃)₂-n-C₄H₉, (CH₂)ₚPh, or (CH₂)ₚOPh;

p is 1-3;

Ph is a phenyl ring, optionally substituted with halogen, trihalogenated methyl, methyl, OR¹³, or C(O)CH₃, where R¹³ is H, CH₃, C₂H₅, C(O)CH₃, or phenyl; and

Halogen is Cl, I, Br, or F.
Preferred compounds of formula I are those wherein:

R¹ is CO₂R or CO₂⁻R⁺;

R is H, CH₃, C₂H₅, or n-C₃H₇;

R⁺ is Na⁺ or NH₄⁺;

G is CH₂;

Z is cis-CH₂CH═CH, cis-CH═CHCH₂, CH₂C≡C, C≡CCH₂, or (CH₂)₃;

A and D are independently CH═CH or C≡C;

X is CHOH;

Yis n-C₅H₁₁, C(CH₃)H-n-C₄H₉, C(CH₃)₂-n-C₄H₉, (CH₂)ₚPh, or (CH₂)ₚOPh;

p is 1-3; and

Ph is a phenyl ring, optionally substituted with chloro, fluoro, methyl, CF₃, C(O)CH₃, OH, or OC(O)CH₃.

Amonng the especially preferred compounds of the present invention are the following:

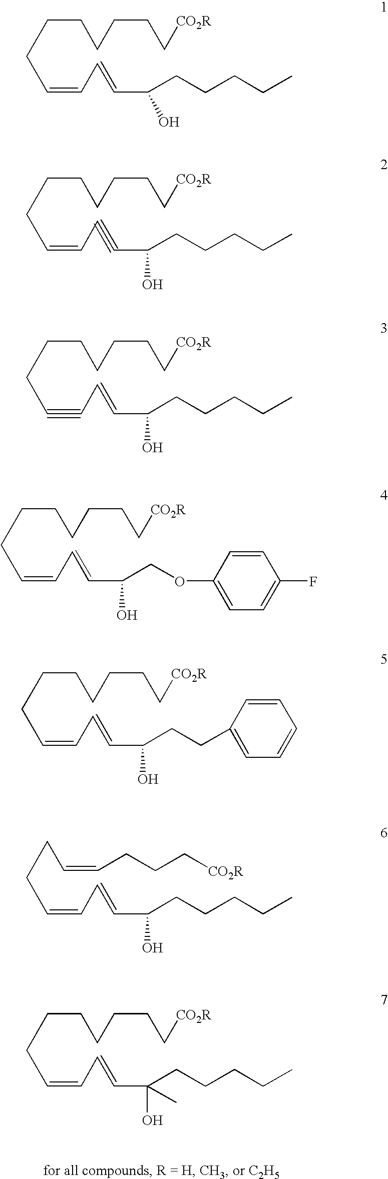

The compounds 13(S)-HODE and its methyl ester (1 with R=H and CH₃, respectively) are commercially available from Biomol Research Company, Plymouth Meeting, Pa. Additionally, 13(S)-HODE, 13(S)-HODE methyl ester, and racemic samples of compound 3 (R=H) are known from J. Med. Chem. 1987, 30(2), 254-264, which is incorporated herein by reference. Other compounds of formula I can be made by the methods illustrated in the following examples 1-7.

### EXAMPLE 1

Synthesis of Compound 1 (R=C₂H₅)

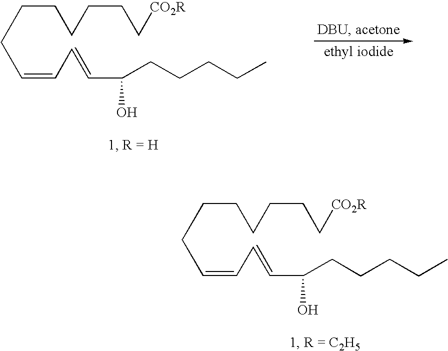

A solution of 13S-HODE (1, R=H), acetone, 1,8-diazabicyclo[5.4.0]undec-7-ene (DBU), and ethyl iodide is stirred for 16 h to provide 13S-HODE ethyl ester (1, R=C₂H₅) after aqueous workup and chromatographic purification.

### EXAMPLE 2

Synthesis of Compounds 2 (R=H, CH₃, and C₂H₅)

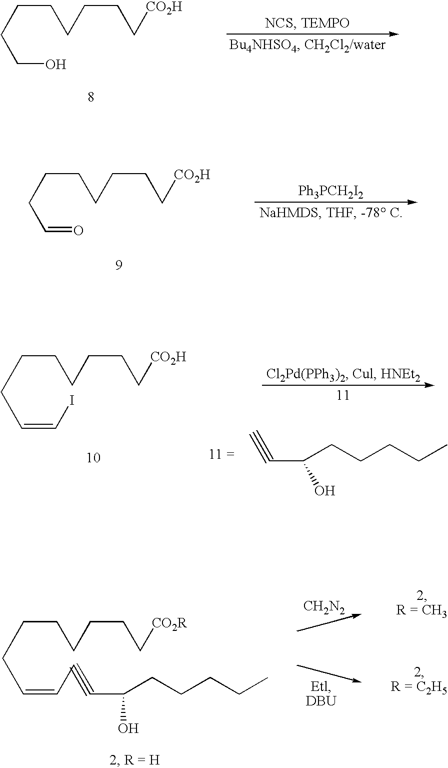

9-Hydroxynonanoic acid (8; commercially available from Matrix Scientific Corporation, Post Office Box 25067, Columbia, S.C. 29224-5067) is oxidized to aldehyde 9 using catalytic 2,2,6,6-tetramethyl-piperidinoxyl free radical (TEMPO) and stoichiometric N-chlorosuccinimide (NCS) in a rapidly stirring mixture of CH₂Cl₂, water, and catalytic n-Bu₄NHSO₄. Conversion of 9 to cis-vinyl iodide 10 is effected using Ph₃PCH₂I₂ and NaN(SiMe₃)₂ (NaHMDS) in THF/HMPA. Sonogishira coupling of 10 with (3S)-1-octyn-3-ol (11; commercially available from Aldrich Chemical Co., Post Office Box 355, Milwaukee, Wis. 53201) is accomplished using in HNEt₂ as solvent using stoichiometric CuI and catalytic Cl₂Pd(PPh₃)₂ provides enyne 2 (R=H). Treatment with CH₂N₂ in diethyl ether affords the methyl ester 2 (R=CH₃), while treatment with Etl and DBU in acetone yields ethyl ester 2 (R=C₂H₅).

### EXAMPLE 3

Synthesis of Compounds 3 (R=H, CH₃, AND C₂H₅)

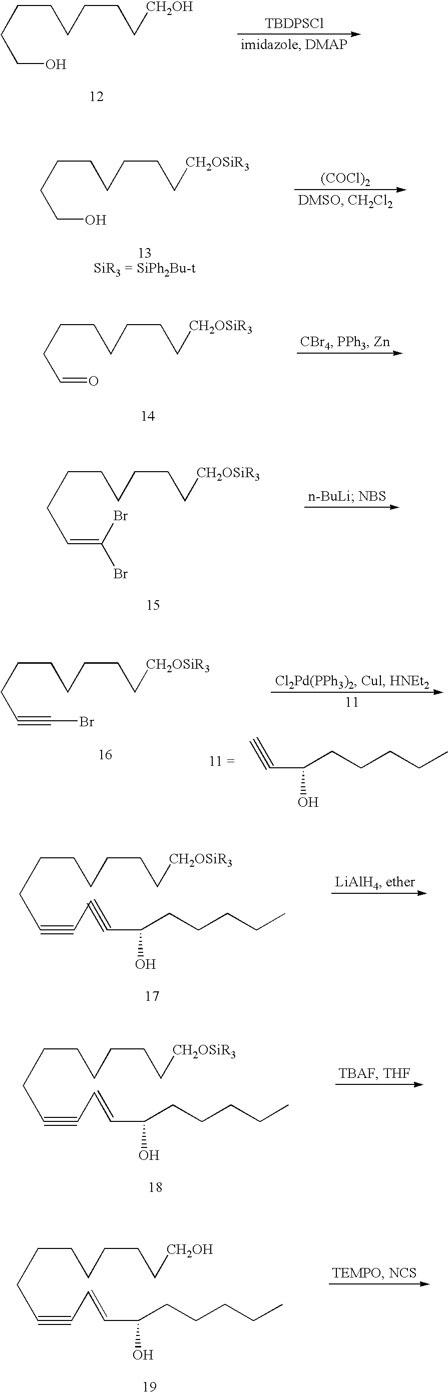

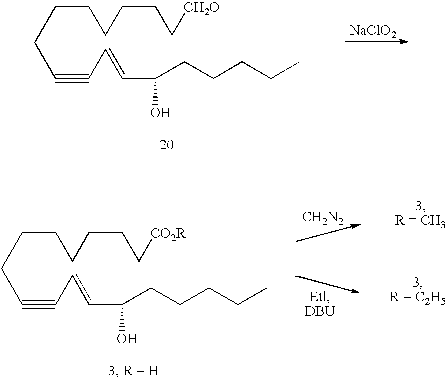

1,9-nonanediol (12) is monoprotected using 0.9 equivalents of t-butyldiphenylchlorosilane in THF containing stoichiometric imidazole and catalytic N,N-dimethylaminopyridine (DMAP), to afford monosilyl ether 13. Swern oxidation using (COCl)₂, dimethylsulfoxide (DMSO), and NEt₃ in CH₂Cl₂ at −78° C. gives aldehyde 14, which is olefinated with CBr₄ in CH₂Cl₂ in the presence of Zn and PPh₃ to give dibromoolefin 15. Metal-halogen exchange in THF at −78° C. is followed by quenching with N-bromosuccinimide (NBS) to afford bromoalkyne 16. Sonogashira coupling with octynol 11 is effected using catalytic Cl₂Pd(PPh₃)₂ and stoichiometric Cul in HNEt₂ solvent to provide diyne 17, which is reduced to trans-ynene 18 with LiAlH₄ in diethyl ether. Silyl ether deprotection with n-Bu₄NF in THF gives diol 19, which is oxidized to aldehyde 20 using stoichiometric NCS and catalytic TEMPO in CH₂Cl₂/water containing catalytic n-Bu₄NHSO₄. Oxidation of 20 to acid 3 (R=H) is effected with NaClO₂ in tert-butanol/water containing saturated aqueous KH₂PO₄ and a THF solution of 2-methyl-2-butene. A solution of acid 3 (R=H) in diethyl ether is treated with diazomethane to afford methyl ester 3 (R=CH₃), while treatment of an acetone solution of acid 3 (R=H) with DBU and ethyl iodide provides ethyl ester 3 (R=C₂H₅).

### EXAMPLE 4

### Synthesis of Compounds 4 (R=H, CH₃, and C₂H₅)

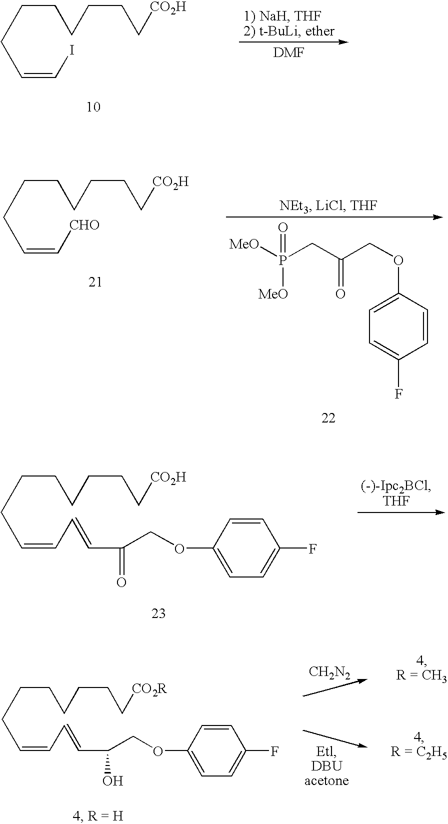

Treatment of vinyl iodide 10 with sodium hydride in THF generates the corresponding sodium carboxylate, which is treated with t-butyllithium in diethyl ether at −78° C. generates a vinyllithium species that is trapped with N,N-dimethylformamide (DMF) to provide cis-enal 21 after workup. Horner-Emmons condensation of 21 with phosphonate 22 [prepared as follows: 1. 2-(p-fluorophenoxy)acetic acid, diazomethane (to form methyl 2-(p-fluorophenoxy)acetate); 2. (MeO)₂P(O)CH₃, n-butyllithium] using NEt₃/LiCl in THF provides dienone 23, which is reduced to R alcohol acid 4 (R=H) using (−)-B-chlorodiisopinocampheylborane [(−)-Ipc₂BCl] in THF at 0° C. Treatment of 4 (R=H) with either diazomethane or ethyl iodide/DBU provides 4 (R=CH₃) and 4 (R=C₂H₅), respectively.

### EXAMPLE 5

Synthesis of Compounds 5 (R=H, CH₃, and C₂H₅)

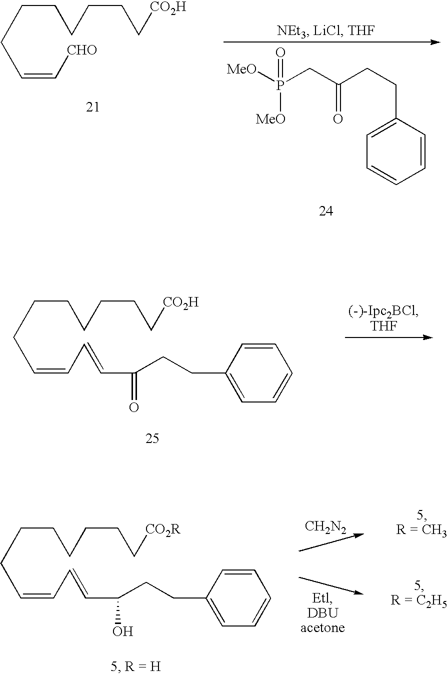

Horner-Emmons condensation of enal 21 with phosphonate 24 [commercially available from Fisher Scientific, 1 Reagent Lane, Fair Lawn, N.J., 07410] using NEt₃/LiCl in THF provides dienone 25, which is reduced to S alcohol acid 5 (R=H) using (−)-Ipc₂BCl in THF at 0° C. Treatment of 5 (R=H) with either diazomethane or ethyl iodide/DBU provides 5 (R=CH₃) or 5 (R=C₂H₅), respectively.

### EXAMPLE 6

Synthesis of Compounds 6 (R=H, CH₃, and C₂H₅)

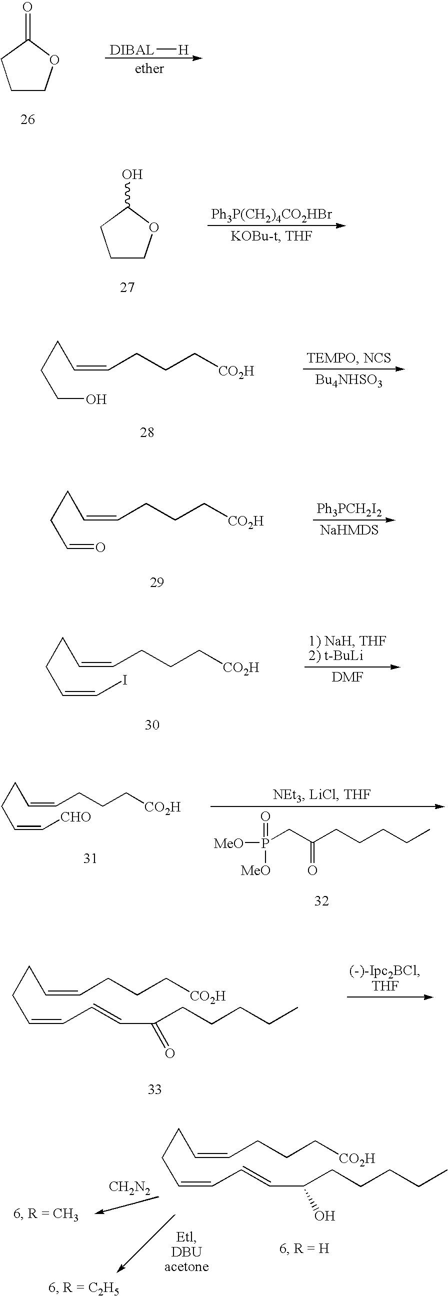

Reduction of γ-butyrolactone (26) with DIBAL-H in diethyl ether at −78° C. provides tetrahydrofuranol 27, which is condensed with Ph₃P⁺(CH₂)₄CO₂H Br (commercially available from Aldrich Chemical Company) in THF in the presence of potassium tert-butoxide to afford olefin 28. Oxidation of 28 with catalytic TEMPO/stoichiometric NCS in rapidly stirring water/CH₂Cl₂ containing catalytic n-Bu₄NHSO₄ yields aldehyde 29, which is treated with Ph₃PCH₂I₂ and NaHMDS in THF/HMPA at −78° C. to give cis-vinyl iodide 30. Treatment of 30 with NaH in THF generates the sodium carboxylate, which is treated sequentially with tert-butyl lithium in diethyl ether at −78° C. and DMF to provide cis-enal 31. Horner-Emmons olefination of 31 with phosphonate 32 (commercially available from Aldrich Chemical Company) in THF in the presence of NEt₃/LiCl gives dienone 33, which is reduced with (−)-Ipc₂BCl in THF at 0° C. to give dienol 6 (R=H). Treatment of 6 (R=H) with either diazomethane or ethyl iodide/DBU provides 6 (R=CH₃) or 6 (R=C₂H₅), respectively.

### EXAMPLE 7

Synthesis of Compounds 7 (R=H, CH₃, and C₂H₅)

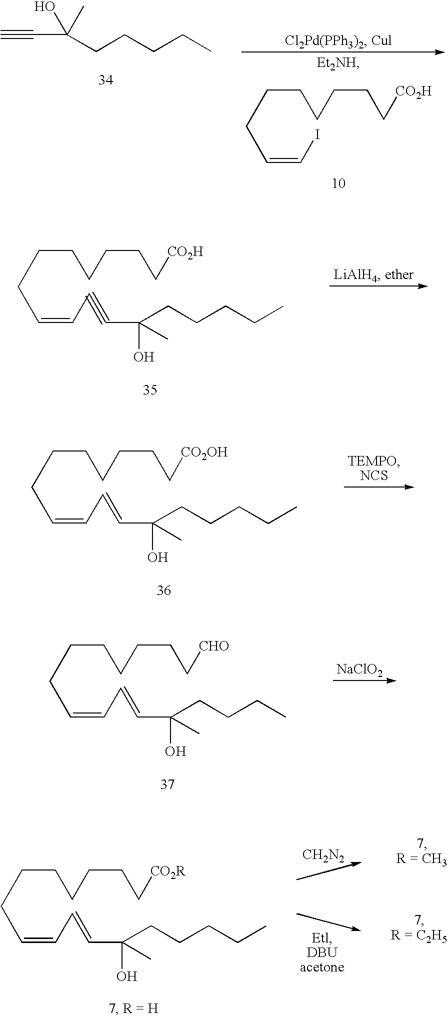

(3RS)-3-Methyl-1-octyn-3-ol (34, commercially available from Lancaster Synthesis Inc., Post Office Box 1000 Windham, N.H., 03087-9977) is coupled with vinyl iodide 10 using catalytic Cl₂Pd(PPh₃)₂ and stoichiometric Cul in HNEt₂ to afford enyne 35, which is reduced to diene diol 36 with LiAlH₄ in diethyl ether. Oxidation to aldehyde 37 is performed using catalytic TEMPO and stoichiometric NCS in CH₂Cl₂/water containing catalytic n-Bu₄NHSO₄. Reaction of 37 with NaClO₂ in tert-butanol/water containing saturated aqueous KH₂PO₄ and a THF solution of 2-methyl-2-butene provides acid 7 (R=H). Treatment of 7 (R=H) with either diazomethane or ethyl iodide/DBU provides 7 (R=CH₃) or 7 (R=C₂H₅), respectively.

According to the methods of the present invention, a compound of formula I is administered in a pharmaceutically acceptable carrier for topical ophthalmic administration. The compositions are formulated in accordance with methods known in the art. The compositions may contain more than one compound of formula I. Additionally, the compositions may contain a second drug, other than a compound of formula I.

The compositions of the present invention contain a pharmaceutically effective amount of a compound of formula I. As used herein, “a pharmaceutically effective amount” means an amount sufficient to reduce or eliminate dry eye symptoms. Generally, the compositions of the present invention will contain from 0.000001% to 0.01% of a compound of formula I. Preferably, the compositions of the present invention will contain from 0.00001% to 0.001%.

The compositions administered according to the present invention may to also include various other ingredients, including but not limited to surfactants, tonicity agents, buffers, preservatives, co-solvents and viscosity building agents.

Various tonicity agents may be employed to adjust the tonicity of the composition, preferably to that of natural tears for ophthalmic compositions. For example, sodium chloride, potassium chloride, magnesium chloride, calcium chloride, dextrose and/or mannitol may be added to the composition to approximate physiological tonicity. Such an amount of tonicity agent will vary, depending on the particular agent to be added. In general, however, the compositions will have a tonicity agent in an amount sufficient to cause the final composition to have an ophthalmically acceptable osmolality (generally about 150-450 mOsm, preferably 250-350 mOsm).

An appropriate buffer system (e.g., sodium phosphate, sodium acetate, sodium citrate, sodium borate or boric acid) may be added to the compositions to prevent pH drift under storage conditions. The particular concentration will vary, depending on the agent employed. Preferably, however; the buffer will be chosen to maintain a target pH within the range of pH 5.5-8.

Other compounds designed to lubricate, “wet,” approximate the consistency of endogenous tears, aid in natural tear build-up, or otherwise provide temporary relief of dry eye symptoms and conditions upon ocular administration to the eye are known in the art and may be included in the compositions of the present invention. Such compounds may enhance the viscosity of the composition, and include, but are not limited to: monomeric polyols, such as, glycerol, propylene glycol, ethylene glycol; polymeric polyols, such as, polyethylene glycol, hydroxypropylmethyl cellulose (“HPMC”), carboxy methylcellulose sodium, hydroxy propylcellulose (“HPC”), dextrans, such as, dextran 70; water soluble proteins, such as gelatin; and vinyl polymers, such as, polyvinyl alcohol, polyvinylpyrrolidone, povidone and carbomers, such as, carbomer 934P, carbomer 941, carbomer 940, carbomer 974P.

Topical ophthalmic products are typically packaged in multidose form. Preservatives are typically required to prevent microbial contamination during use. Suitable preservatives include: benzalkonium chloride, chlorobutanol, benzododecinium bromide, methyl paraben, propyl paraben, phenylethyl alcohol, edetate disodium, sorbic acid, polyquaternium-1, or other agents known to those skilled in the art. Such preservatives are typically employed at a level of from 0.001 to 1.0% w/v. Unit dose compositions of the present invention will be sterile, but typically will not contain a preservative and will be unpreserved.

Generally, 1-2 drops of such compositions will be administered from once to many times per day.

Representative eye drop formulations are provided in Examples 1 and 2 below.

### EXAMPLE 8

| Ingredient | Concentration (% w/v) |
|---|---|
| Compound of formula I | 0.00001-0.001 |
| Polyoxyl 40 Hydrogenated Castor Oil | 0.1-0.5 |
| Tromethamine | 0.05-0.15 |
| Boric Acid | 0.1-0.3 |
| Mannitol | qs 280-310 mOsm |
| Edetate Disodium | 0.01 |
| Benzalkonium Chloride | 0.01-0.015 |
| NaOH/HCl | q.s. to pH 6-8 |
| Purified Water | q.s. to 100 |

### EXAMPLE 9

| Ingredient | Concentration (% w/v) |
|---|---|
| Compound of formula I | 0.00003 |
| Hydroxypropyl methylcellulose | 0.1-0.5 |
| Dextran 70 | 0.1 |
| Sodium Chloride | 0.8 |
| Potassium Chloride | 0.12 |
| Dibasic Sodium Phosphate | 0.025 |
| Edetate Disodium | 0.01 |
| Polyquaternium-1 | 0.001-0.005 |
| NaOH/HCl | q.s. to pH 6-8 |
| Purified Water | q.s. to 100 |

This invention has been described by reference to certain preferred embodiments; however, it should be understood that it may be embodied in other specific forms or variations thereof without departing from its special or essential characteristics. The embodiments described above are therefore considered to be illustrative in all respects and not restrictive, the scope of the invention being indicated by the appended claims rather than by the foregoing description.

What is claimed is:

1. A method for the treatment of dry eye in a mammal, which comprises topically administering to the eye of the mammal a composition comprising a pharmaceutically acceptable carrier and a pharmaceutically effective amount of a compound of formula I: 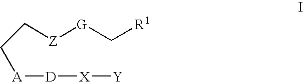
R¹ is CO₂R, CH₂OR², CONR³R⁴, or CO₂⁻R⁺;
R is H, C₁-C₆ alkyl, C₃-C₆ alkenyl, C₃-C₆ alkynyl, C₃-C₆ cycloalkyl, or phenyl;
R⁺ is Li⁺, Na⁺, K⁺, or an ammonium moiety of formula ⁺NR⁵R⁶R⁷R⁸, where R⁵, R⁶, R⁷, and R⁸ are independently H or C₁-C₆ alkyl, each alkyl group optionally bearing an OH or OCH₃ substituent;
R² is H, C(O)R⁹, C₁-C₆ alkyl, C₃-C₆ alkenyl, C₃-C₆ alkynyl, C₃-C₆ cycloalkyl, benzyl, or phenyl, where R⁹ is H, C₁₋₆ alkyl, C₃-C₆ alkenyl, C₃-C₆ alkynyl, C₃-C₆ cycloalkyl, benzyl, or phenyl;
R³ and R⁴ are independently H, C₁-C₆ alkyl, C₃-C₆ alkenyl, C₃-C₆ alkynyl, C₃-C₆ cycloalkyl, H, benzyl, phenyl, OH, OCH₃, or OC₂H₅, provided that at most only one of R³ and R⁴ is OH, OCH₃, or OC₂H₅;
G is CH₂, O, or S;
Z is CH₂CH═CH, CH═CHCH₂, CH₂C≡C, C≡CCH₂, (CH₂)₃, or CH═C═CH when G is CH₂, and is CH═CHCH₂, C≡CCH₂, or (CH₂)₃ when G is O or S;
A and D are independently CH₂CH₂, CH═CH, or C≡C, provided that if A is CH₂CH₂, then D is not CH₂CH₂;
X is C═O or CR¹⁰R₁₁;
R¹⁰ is H or CH₃;
R¹¹ is H, C₁-C₆ alkyl, C₃-C₆ alkenyl, C₃-C₆ alkynyl, C₃-C₆ cycloalkyl, phenyl, benzyl, or C(O)R¹², where R¹² is H, C₁-C₆ alkyl, C₃-C₆ alkenyl, C₃-C₆ alkynyl, C₃-C₆ cycloalkyl, phenyl, or benzyl;
Y is n-C₅H₁₁, C(CH₃)H-n-C₄H₉, C(CH₃)₂-n-C₄H₉, (CH₂)ₚPh, or (CH₂)ₚOPh;
p is 1-3;
Ph is a phenyl ring, optionally substituted with halogen, trihalogenated methyl, methyl, OR¹³, or C(O)CH₃, where R¹³ is H, CH₃, C₂H₅, C(O)CH₃, or phenyl; and
Halogen is Cl, I, Br, or F.

2. The method of claim 1, wherein for the compound of formula I:
R¹ is CO₂R or CO₂⁻R⁺;
R is H, CH₃, C₂H₅, or n-C₃H₇;
R⁺ is Na⁺ or NH₄⁺;
G is CH₂;
Z is cis-CH₂CH═CH, cis-CH═CHCH₂, CH₂C≡E, C≡CCH₂, or (CH₂)₃;
A and D are independently CH═CH or C≡C;
X is CHOH;
Y is n-C₅H₁₁, C(CH₃)H-n-C₄H₉, C(CH₃)₂-n-C₄H₉, (CH₂)ₚPh, or (CH₂)ₚOPh;
p is 1-3; and
Ph is a phenyl ring, optionally substituted with chloro, fluoro, methyl, CF₃, C(O)CH₃, OH, or OC(O)CH₃.

3. The method of claim 2, wherein the compound of formula I is selected from the group consisting of: 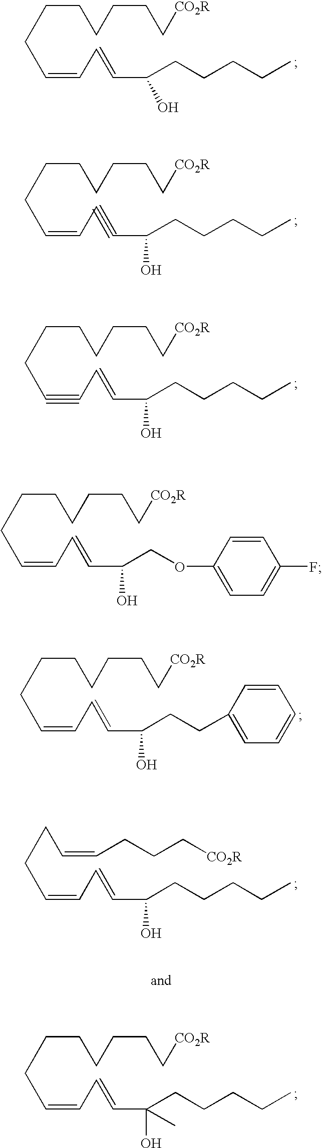
where for all compounds, R=H, CH₃, or C₂H₅.

4. The method of claim 1 wherein the pharmaceutically acceptable amount is from 0.000001% to 0.01% (w/w).

5. The method of claim 4 wherein the pharmaceutically acceptable amount is from 0.00001 % to 0.001% (w/v).

6. The method of claim 1 wherein the pharmaceutically acceptable carrier comprises one or more ingredients selected from the group consisting of surfactants; tonicity agents; buffers; preservatives; co-solvents; and viscosity building agents.

7. A composition for the treatment of dry eye in humans comprising a pharmaceutically acceptable carrier and a pharmaceutically effective amount of one or more compounds of formula I: 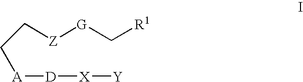
wherein:
R¹ is CO₂R, CH₂OR², CONR³R⁴, or CO₂⁻R⁺;
R is H, C₁-C₆ alkyl, C₃-C₆ alkenyl, C₃-C₆ alkynyl, C₃-C₆ cycloalkyl, or phenyl;
R⁺ is Li⁺, Na⁺, K⁺, or an ammonium moiety of formula ⁺NR⁵R⁶R⁷R⁸, where R⁵, R⁶, R⁷, and R⁸ are independently H or C₁-C₆ alkyl, each alkyl group optionally bearing an OH or OCH₃ substituent;
R² is H, C(O)R⁹, C₁-C₆ alkyl, C₃-C₆ alkenyl, C₃-C₆ alkynyl, C₃-C₆ cycloalkyl, benzyl, or phenyl, where R⁹ is H, C₁₋₆ alkyl, C₃-C₆ alkenyl, C₃-C₆ alkynyl, C₃-C₆ cycloalkyl, benzyl, or phenyl;
R³ and R⁴ are independently H, C₁-C₆ alkyl, C₃-C₆ alkenyl, C₃-C₆ alkynyl, C₃-C₆ cycloalkyl, H, benzyl, phenyl, OH, OCH₃, or OC₂H₅, provided that at most only one of R³ and R⁴ is OH, OCH₃, or OC₂H₅;
G is CH₂, O, or S;
Z is CH₂CH═CH, CH═CHCH₂, CH₂C≡C, C≡CCH₂, (CH₂)₃, or CH═C═CH when G is CH₂, and is CH═CHCH₂, C≡CCH₂, or (CH₂)₃ when G is O or S;
A and D are independently CH₂CH₂, CH═CH, or C≡C, provided that if A is CH₂CH₂, then D is not CH₂CH₂;
X is C═O or CR¹⁰R₁₁;
R¹⁰ is H or CH₃;
R¹¹ is H, C₁-C₆ alkyl, C₃-C₆ alkenyl, C₃-C₆ alkynyl, C₃-C₆ cycloalkyl, phenyl, benzyl, or C(O)R¹², where R¹² is H, C₁-C₆ alkyl, C₃-C₆ alkenyl, C₃-C₆ alkynyl, C₃-C₆ cycloalkyl, phenyl, or benzyl;
Y is n-C₅H₁₁, C(CH₃)H-n-C₄H₉, C(CH₃)₂-n-C₄H₉, (CH₂)ₚPh, or (CH₂)ₚOPh;
p is 1-3;
Ph is a phenyl ring, optionally substituted with halogen, trihalogenated methyl, methyl, OR¹³, or C(O)CH₃, where R¹³ is H, CH₃, C₂H₅, C(O)CH₃, or phenyl; and
Halogen is Cl, I, Br, or F.

8. The composition of claim 7, wherein for the compound of formula l:
R¹ is CO₂R or CO₂⁻R⁺;
R is H, CH₃, C₂H₅, or n-C₃H₇;
R⁺ is Na⁺ or NH₄⁺;
G is CH₂;
Z is cis-CH₂CH═CH, cis-CH═CHCH₂, CH₂C≡C, C≡CCH₂, or (CH₂)₃;
A and D are independently CH═CH or C≡C;
X is CHOH;
Y is n-C₅H₁₁, C(CH₃)H-n-C₄H₉, C(CH₃)₂-n-C₄H₉, (CH₂)ₚPh, or (CH₂)ₚOPh;
p is 1-3; and
Ph is a phenyl ring, optionally substituted with chloro, fluoro, methyl, CF₃, C(O)CH₃, OH, or OC(O)CH₃.

9. The composition of claim 8, wherein the compound of formula I is selected from the group consisting of: 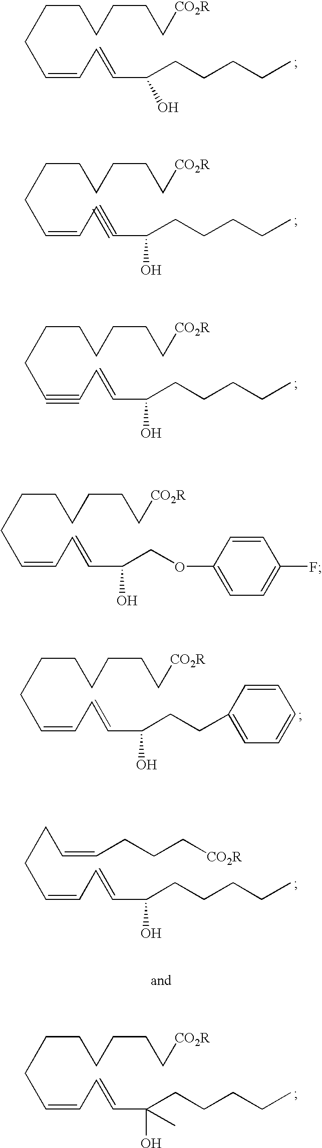
where for all compounds, R=H, CH₃, or C₂H₅.

10. The composition of claim 7, wherein the composition is a topical ophthalmic formulation.

11. The composition of claim 8, wherein the composition is a topical ophthalmic formulation.

12. The composition of claim 9, wherein the composition is a topical ophthalmic formulation.

13. A compound of formula I: 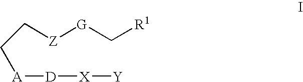
wherein:
R¹ is CO₂R, CH₂OR², CONR³R⁴, or CO²⁻R⁺;
R is H, C₁-C₆ alkyl, C₃-C₆ alkenyl, C₃-C₆ alkynyl, C₃-C₆ cycloalkyl, or phenyl;
R⁺ is Li⁺, Na⁺, K⁺, or an ammonium moiety of formula ⁺NR⁵R⁶R⁷R⁸, where R⁵, R⁶, R⁷, and R⁸ are independently H or C₁-C₆ alkyl, each alkyl group optionally bearing an OH or OCH₃ substituent;
R² is H, C(O)R⁹, C₁-C₆ alkyl, C₃-C₆ alkenyl, C₃-C₆ alkynyl, C₃-C₆ cycloalkyl, benzyl, or phenyl, where R⁹ is H, C₁₋₆ alkyl, C₃-C₆ alkenyl, C₃-C₆ alkynyl, C₃-C₆ cycloalkyl, benzyl, or phenyl;
R³ and R⁴ are independently H, C₁-C₆ alkyl, C₃-C₆ alkenyl, C₃-C₆ alkynyl, C₃-C₆ cycloalkyl, H, benzyl, phenyl, OH, OCH₃, or OC₂H₅, provided that at most only one of R³ and R⁴ is OH, OCH₃, or OC₂H₅;
G is CH₂, O, or S;
Z is CH₂CH═CH, CH═CHCH₂, CH₂C≡C, C≡CCH₂, (CH₂)₃, or CH═C═CH when G is CH₂, and is CH═CHCH₂, C≡CCH₂, or (CH₂)₃ when G is O or S;
A and D are independently CH₂CH₂, CH═CH, or C≡C, provided that if A is CH₂CH₂, then D is not CH₂CH₂;
X is C═O or CR¹⁰R₁₁;
R¹⁰ is H or CH₃;
R¹¹ is H, C₁-C₆ alkyl, C₃-C₆ alkenyl, C₃-C₆ alkynyl, C₃-C₆ cycloalkyl, phenyl, benzyl, or C(O)R¹², where R¹² is H, C₁-C₆ alkyl, C₃-C₆ alkenyl, C₃-C₆ alkynyl, C₃-C₆ cycloalkyl, phenyl, or benzyl;
Y is n-C₅H₁₁, C(CH₃)H-n-C₄H₉, C(CH₃)₂-n-C₄H₉, (CH₂)ₚPh, or (CH₂)ₚOPh;
p is 1-3;
Ph is a phenyl ring, optionally substituted with halogen, trihalogenated methyl, methyl, OR¹³, or C(O)CH₃, where R¹³ is H, CH₃, C₂H₅, C(O)CH₃, or phenyl; and
Halogen is Cl, I, Br, or F;
with the proviso that the following compounds are excluded: 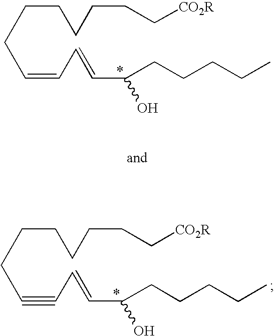
where R=H, C₁-C₆ alkyl, C₃-C₆ alkenyl, C₃-C₆ alkynyl, C₃-C₆ cycloalkyl, or phenyl; or R is a carboxylate salt of formula CO₂⁻R⁺, where R⁺ is Li⁺, Na⁺, K⁺, or an ammonium moiety of formula ⁺NR⁵R⁶R⁷R⁸, where R⁵, R⁶, R⁷, and R⁸ are independently H or C₁-C₆ alkyl, each alkyl group optionally bearing an OH or OCH₃ substituent; and with the substituents at the hydroxyl-bearing carbon (*) being arranged to afford either the R or S absolute configuration.

14. A compound of claim 13, wherein:
R¹ is CO₂R or CO₂⁻R⁺;
R is H, CH₃, C₂H₅, or n-C₃H₇;
R⁺ is Na⁺ or NH₄⁺;
G is CH₂;
Z is cis-CH₂CH═CH, cis-CH═CHCH₂, CH₂C≡C, C≡CCH₂, or (CH₂)₃;
A and D are independently CH═CH or C≡E;
X is CHOH;
Y is n-C₅H₁₁, C(CH₃)H-n-C₄H₉, C(CH₃)₂-n-C₄H₉, (CH₂)ₚPh, or (CH₂)ₚOPh;
p is 1-3; and
Ph is a phenyl ring, optionally substituted with chloro, fluoro, methyl, CF₃, C(O)CH₃, OH, or OC(O)CH₃.

15. A compound of claim 14, selected from the group consisting of: 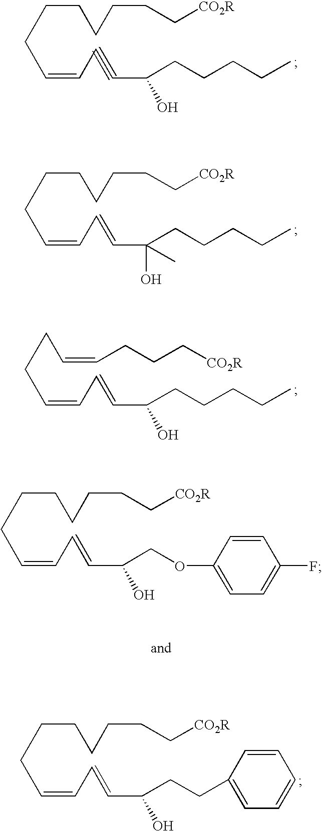
where R=H, CH₃, or C₂H₅.
